(19) United States
(12) Patent Application Publication
(10) Pub. No.: US 2020/0376193 A1
(43) Pub. Date: Dec. 3, 2020

(54) PUNCTURE MEMBER DETACHMENT DETECTION APPARATUS, PUNCTURE MEMBER DETACHMENT DETECTION SYSTEM, AND PUNCTURE MEMBER DETACHMENT DETECTION METHOD

(71) Applicant: NEC Platforms, Ltd., Kawasaki-shi, Kanagawa (JP)

(72) Inventor: Shinichi MIYAZAKI, Kanagawa (JP)

(73) Assignee: NEC Platforms, Ltd., Kawasaki-shi, Kanagawa (JP)

(21) Appl. No.: 16/960,703

(22) PCT Filed: Nov. 16, 2018
(86) PCT No.: PCT/JP2018/042506

§ 371 Date: Jul. 8, 2020

(30) Foreign Application Priority Data
Jan. 9, 2018 (JP) 2018-001118

(51) Int. Cl. A61M 5/158; A61M 5/168

(52) U.S. Cl. CPC A61M 5/158; A61M 5/168; A61M 2230/50; A61M 2230/06; A61M 2205/18

(57) ABSTRACT

A puncture member detachment detection apparatus (1) includes a puncture member (10) that punctures a living body, and living body contact detection means (20) for detecting contact with the living body. The living body contact detection means (20) is provided at a part of the puncture member (10) that comes into contact with the living body. When the living body contact detection means (20) detects that the contact with the living body is released, it notifies an alarm transmitter (30) that the contact with the living body is released. Thus, it is possible to provide a puncture member detachment detection apparatus capable of preventing an erroneous detection as to whether a puncture member is detached.

### TECHNICAL FIELD

The present invention relates to a puncture member detachment detection apparatus, a puncture member detachment detection system, and a puncture member detachment detection method, and in particular, to a puncture member detachment detection apparatus, a puncture member detachment detection system, and a puncture member detachment detection method that detect that a puncture member such as an injection needle is detached from a living body such as a human body.

### BACKGROUND ART

An injection needle may be detached while a patient himself/herself is not aware of it, for example, when an infusion is carried out. Further, for example, when a patient has dementia, the patient may remove an injection needle by himself/herself. Accordingly, it is necessary for an attendant or the like to watch a patient having dementia, and the attendant may have to watch him/her on a 24-hour basis. Therefore, an apparatus capable of automatically detecting a detachment of a puncture member such as an injection needle is demanded.

Patent Literature 1 discloses an extravasation detection apparatus including a blood flow noise sound generation unit for generating a blood flow noise sound and a reception unit such as a microphone that collects the blood flow noise sound. In Patent Literature 1, the blood flow noise sound generation unit is provided near a tip opening of a catheter, and the reception unit is attached and fixed on the skin near the tip opening of the catheter. Then, in Patent Literature 1, when the reception unit cannot receive a normal blood flow noise sound and detects a sound different from the normal blood flow noise sound in the extravasation detection apparatus, it is determined that the tip opening of the catheter is detached from a blood vessel.

### CITATION LIST

### Patent Literature

### Patent Literature 1: Japanese Unexamined Patent Application Publication No. 2011-200429

### SUMMARY OF INVENTION

### Technical Problem

However, in the technique disclosed in Patent Literature 1, even in a state in which the tip opening of the catheter is left in the blood vessel, the reception unit detects a sound different from the normal blood flow noise sound if the reception unit is detached. In such a case, the extravasation detection apparatus erroneously determines that the tip opening of the catheter is detached from the blood vessel. Further, in the technique disclosed in Patent Literature 1, even in state in which the tip opening of the catheter is detached from the blood vessel, the reception unit may pick up an external noise sound in the same range as that of the normal blood flow noise sound. In such a case, the extravasation detection apparatus erroneously determines that the tip opening of the catheter is left in the blood vessel. That is, the technique disclosed in Patent Literature 1 has a problem that it may be erroneously determined as to whether the tip opening of the catheter is detached from the blood vessel.

The present disclosure has been made to solve the above-described problem and an object thereof is to provide a puncture member detachment detection apparatus, a puncture member detachment detection system, and a puncture member detachment detection method that are capable of preventing an erroneous detection as to whether a puncture member is detached.

### Solution to Problem

A puncture member detachment detection apparatus according to a first aspect of the present disclosure includes: a puncture member that punctures a living body; and living body contact detection means for detecting contact with the living body, in which the living body contact detection means is provided at a part of the puncture member that comes into contact with the living body.

A puncture member detachment detection system according to a second aspect of the present disclosure includes a puncture member detachment detection apparatus and an alarm transmitter, in which the puncture member detachment detection apparatus includes: a puncture member that punctures a living body; and living body contact detection means for detecting contact with the living body, the living body contact detection means is provided at a part of the puncture member that comes into contact with the living body, and the alarm transmitter transmits an alarm when the living body contact detection means detects that the contact with the living body is released.

A puncture member detachment detection method according to a third aspect of the present disclosure includes: detecting, by living body contact detection means provided at a part of a puncture member that comes into contact with a living body, contact with the living body; and notifying an alarm transmitter that the contact with the living body is released when the living body contact detection means detects that the contact with the living body is released.

### Advantageous Effects of Invention

According to the present invention, it is possible to provide a puncture member detachment detection apparatus, a puncture member detachment detection system, and a puncture member detachment detection method that are capable of preventing an erroneous detection as to whether a puncture member is detached.

### BRIEF DESCRIPTION OF DRAWINGS

FIG. 1 is a diagram showing an example of a configuration of a puncture member detachment detection apparatus according to a first example embodiment;

FIG. 2 is a diagram showing another example of the configuration of the puncture member detachment detection apparatus according to the first example embodiment;

FIG. 3 is a diagram showing a configuration example of a puncture member detachment detection system according to the first example embodiment;

FIG. 4 is a diagram for explaining an installation example of a living body contact detection unit when a puncture member according to the first example embodiment is a wing-like needle;

FIG. 5 is a diagram for explaining an installation example of the living body contact detection unit when the puncture member according to the first example embodiment is a wing-like needle;

FIG. 6 is a diagram for explaining an installation example of the living body contact detection unit when the puncture member according to the first example embodiment is an indwelling needle;

FIG. 7 is a diagram for explaining an installation example of the living body contact detection unit when the puncture member according to the first example embodiment is an indwelling needle;

FIG. 8 is a flowchart showing an operation example of the puncture member detachment detection apparatus according to the first example embodiment; and

FIG. 9 is a block diagram showing a configuration example of a heart rate sensor according to a second example embodiment.

### DESCRIPTION OF EMBODIMENTS

Example embodiments according to the present invention will be described hereinafter in detail with reference to the drawings. The same or corresponding elements are denoted by the same symbols throughout the drawings, and repeated descriptions are omitted as necessary for the sake of clarity.

### First Example Embodiment

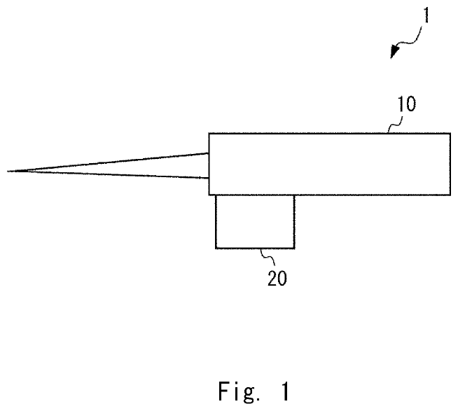

First, a configuration example of a puncture member detachment detection apparatus 1 according to a first example embodiment of the present invention is described with reference to FIG. 1. The puncture member detachment detection apparatus 1 includes a puncture member 10 and a living body contact detection unit 20.

The puncture member 10 is a puncture member that punctures a living body, such as an injection needle, a catheter, a drain tube, and the like.

The living body contact detection unit 20 detects contact with a living body. Further, the living body contact detection unit 20 is provided at a part of the puncture member 10 that comes into contact with the living body. Further, when the living body contact detection unit 20 detects that the contact with the living body is released, the living body contact detection unit 20 notifies an alarm transmitter 30 that the contact with the living body is released. Note that the alarm transmitter 30 may be provided in the puncture member detachment detection apparatus 1 or may instead be provided separately from the puncture member detachment detection apparatus 1.

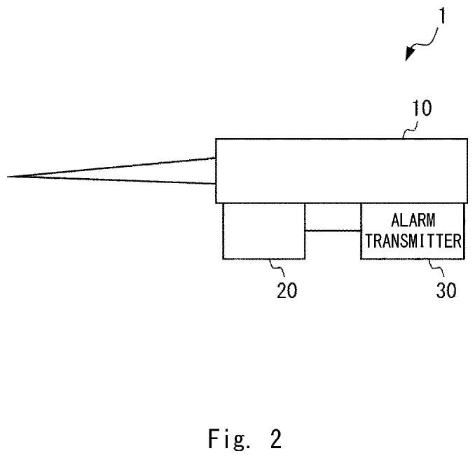

FIG. 2 shows an example in which the puncture member detachment detection apparatus 1 includes the alarm transmitter 30. In the example of FIG. 2, the alarm transmitter 30 is provided on the puncture member 10. Further, the alarm transmitter 30 is connected to the living body contact detection unit 20 wirelessly or through a wire. Note that a location where the alarm transmitter 30 is provided may not be on the puncture member 10.

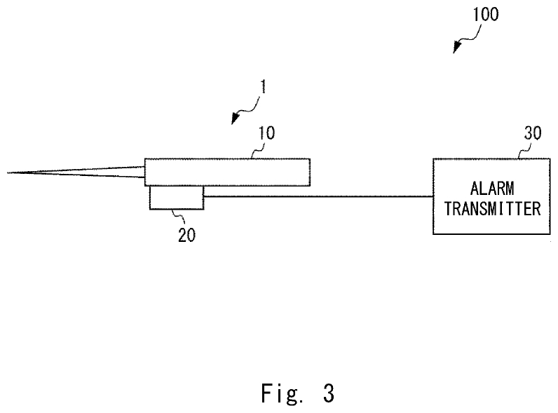

FIG. 3 shows an example in which the alarm transmitter 30 is provided separately from the puncture member detachment detection apparatus 1. Note that the configuration including the puncture member detachment detection apparatus 1 and the alarm transmitter 30 as shown in FIG. 3 is also referred to as a puncture member detachment detection system 100. The alarm transmitter 30 is connected to the living body contact detection unit 20 wirelessly or through a wire. In this example, the alarm transmitter 30 may be provided, for example, in a nurse center. Further, the alarm transmitter 30 may be provided as an application of a smartphone or the like.

Note that the living body contact detection unit 20 may continue to output a detection signal to the alarm transmitter 30 when the contact with the living body is being detected. That is, the living body contact detection unit 20 may stop outputting a detection signal to the alarm transmitter 30 when the contact with the living body is released. In this case, the alarm transmitter 30 transmits an alarm when it no longer receives the detection signal. Further, when the living body contact detection unit 20 detects that the contact with the living body is released, it may output a detection signal indicating that the contact with the living body is released to the alarm transmitter 30. In this case, the alarm transmitter 30 transmits an alarm when it receives the detection signal.

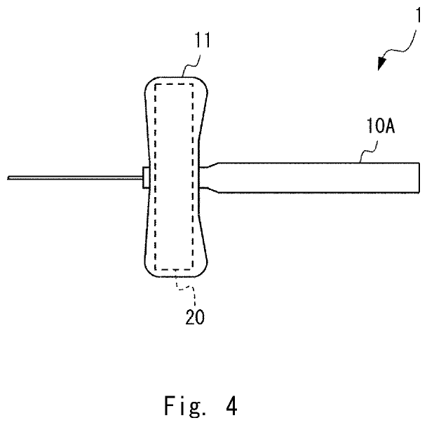

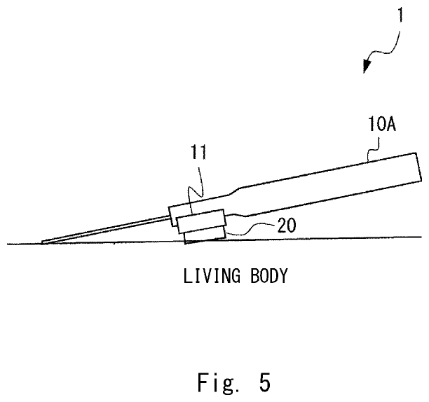

Next, an installation example of the living body contact detection unit 20 when the puncture member 10 is a wing-like needle 10A is described with reference to FIGS. 4 and 5. As shown in FIGS. 4 and 5, the wing-like needle 10A has a wing-like part 11. As shown in FIG. 5, the living body contact detection unit 20 is provided at a part of the wing-like part 11 that comes into contact with the living body. That is, in a state in which the tip of the wing-like needle 10A punctures the living body, the living body contact detection unit 20 is brought into a state in which it is in contact with the living body.

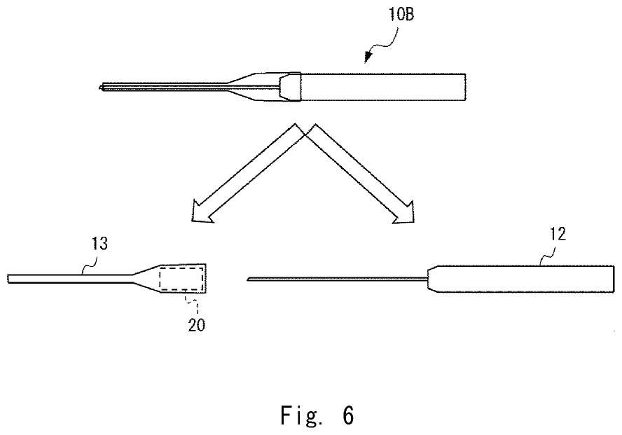

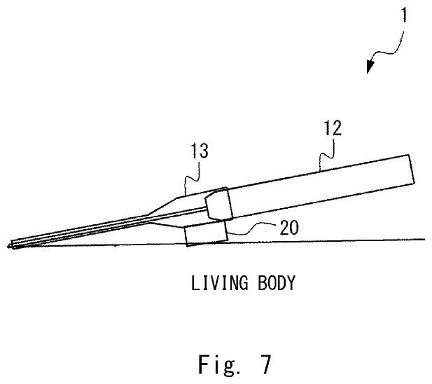

Next, an installation example of the living body contact detection unit 20 when the puncture member 10 is an indwelling needle 10B is described with reference to FIGS. 6 and 7. As shown in FIGS. 6 and 7, the indwelling needle 10B has an inner needle part 12 and an outer cylinder part 13. As shown in FIG. 7, the living body contact detection unit 20 is provided at a part of the outer cylinder part 13 that comes into contact with the living body. That is, in a state in which the outer cylinder part 13 of the indwelling needle 10B is left in the body, the living body contact detection unit 20 is brought into a state in which it is in contact with the living body.

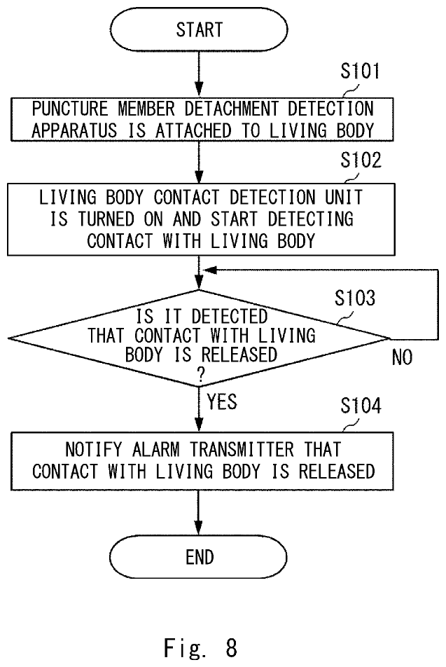

Next, an operation example of the puncture member detachment detection apparatus 1 is described with reference to the flowchart shown in FIG. 8.

First, the puncture member detachment detection apparatus 1 is attached to a living body (Step S101). Note that the puncture member detachment detection apparatus 1 being attached to a living body means that the puncture member 10 punctures the living body and the living body contact detection unit 20 is brought into a state in which it is in contact with the living body.

Next, the puncture member detachment detection apparatus 1 is caused to turn on the operation of the living body contact detection unit 20 and starts detecting contact with the living body (Step S102). Note that the operation of the living body contact detection unit 20 is turned on at the time of starting an infusion, for example, in the case of an application in which a liquid medicine is infused into the living body using the puncture member 10.

Next, the puncture member detachment detection apparatus 1 determines whether the living body contact detection unit 20 detects that the contact with the living body is released (Step S103).

When the puncture member detachment detection apparatus 1 determines that the living body contact detection unit 20 does not detect that the contact with the living body is released (NO in Step S103), the puncture member detachment detection apparatus 1 continues the detection by the living body contact detection unit 20. On the other hand, when the puncture member detachment detection apparatus 1 determines that the living body contact detection unit 20 detects that the contact with the living body is released (YES in Step S103), the puncture member detachment detection apparatus 1 notifies the alarm transmitter 30 that the contact with the living body is released (Step S104).

Then, when the infusion is successfully completed, the puncture member detachment detection apparatus 1 is caused to turn off the operation of the living body contact detection unit 20 and finishes detecting the contact with the living body.

As described above, the puncture member detachment detection apparatus 1 according to the first example embodiment of the present invention includes the puncture member 10 that punctures a living body and the living body contact detection unit 20 that detects contact with the living body. Further, in the puncture member detachment detection apparatus 1, the living body contact detection unit 20 is provided at a part of the puncture member 10 that comes into contact with the living body. Thus, in the puncture member detachment detection apparatus 1 according to the first example embodiment, it is possible to detect that the contact with the living body is released by separating the living body contact detection unit 20 from the living body when the puncture member 10 is detached from the living body. Further, in the puncture member detachment detection apparatus 1, in a state in which the puncture member 10 punctures the living body, the living body contact detection unit 20 does not detect that the contact with the living body is released. Further, in the puncture member detachment detection apparatus 1, it does not happen that the living body contact detection unit 20 fails to detect that the contact with the living body is released in a state in which the puncture member 10 is detached from the living body. That is, the puncture member detachment detection apparatus 1 according to the first example embodiment makes it possible to prevent an erroneous detection as to whether the puncture member 10 is detached.

Accordingly, the use of the puncture member detachment detection apparatus 1 eliminates the need for a person to perform watching of patients. That is, the load of a person who performs watching of patients can be reduced. Further, it is possible to prevent omission of checks due to dozing by persons that is expected in the case of 24-hour watching or the like. Further, it is possible to prevent an accident by early detection of the detachment of the puncture member 10.

### Second Example Embodiment

Next, a puncture member detachment detection apparatus 2 according to a second example embodiment is described. The puncture member detachment detection apparatus 2 according to the second example embodiment uses a heart rate sensor 21 as the living body contact detection unit 20. That is, the puncture member detachment detection apparatus 2 includes the puncture member 10 and the heart rate sensor 21. Note that as the configuration of the puncture member detachment detection apparatus 2 is the same as that of the puncture member detachment detection apparatus 1 according to the first example embodiment shown in FIG. 1, the illustration and the description thereof are omitted.

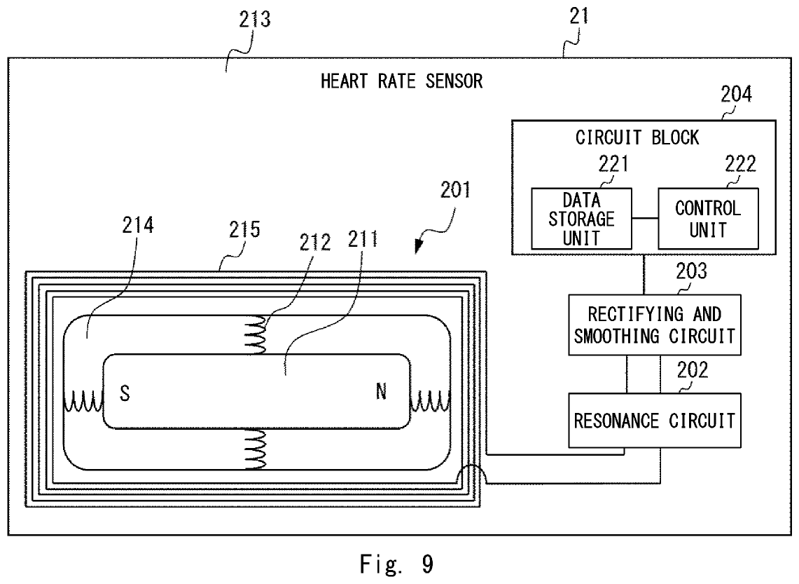

Next, an example of the configuration of the heart rate sensor 21 is described with reference to the block diagram shown in FIG. 9. The heart rate sensor 21 detects that the contact with the living body is released when the heart rate of the living body cannot be measured. The heart rate sensor 21 includes a sensor unit 201, a resonance circuit 202, a rectifying and smoothing circuit 203, and a circuit block 204.

The circuit block 204 includes a data storage unit 221 and a control unit 222. Note that the data storage unit 221 is composed of a Random Access Memory (RAM), a Read Only Memory (ROM), and the like, and stores an operation program and control data of the control unit 222. Further, the control unit 222 is composed of a processor and the like and operates according to a program stored in the data storage unit 221.

The sensor unit 201 is composed of a thin plate-like permanent magnet 211 and a plurality of elastic members 212 that support the permanent magnet 211. Note that the elastic member 212 is, for example, a spring. Further, the permanent magnet 211 is supported in an opening 214 formed in a substrate 213 such as a resin substrate.

An antenna 215 is formed in a coil shape around the opening 214 in the substrate 213. Further, the resonance circuit 202 and the rectifying and smoothing circuit 203 are connected to the antenna 215.

The weight of the permanent magnet 211, and the elastic constant, the number, and the arrangement of the elastic members 212 are set so that the permanent magnet 211 resonates with the vibration of the body surface caused by the human heartbeat or the vibration caused by respiration when the heart rate sensor 21 is brought into contact with the living body. For example, the weight of the permanent magnet 211, and the elastic constant, the number, and the arrangement of the elastic members 212 are set so that the resonance frequency becomes about 50 Hz to 80 Hz. Further, the resonance frequency of the resonance circuit 202 is also set to be about 50 Hz to 80 Hz.

The output of the rectifying and smoothing circuit 203 is supplied to the circuit block 204 as a detection signal. The control unit 222 of the circuit block 204 controls as to whether to cause the alarm transmitter 30 to transmit an alarm in accordance with the detection signal from the rectifying and smoothing circuit 203. Specifically, the control unit 222 outputs the detection signal to the alarm transmitter 30 while the detection signal is being supplied from the rectifying and smoothing circuit 203. Further, when the detection signal is not being supplied from the rectifying and smoothing circuit 203, the control unit 222 does not output the detection signal to the alarm transmitter 30. That is, when the detection signal is not being supplied from the rectifying and smoothing circuit 203, the control unit 222 detects that the contact with the living body is released and stops outputting the detection signal. In this way, the control unit 222 notifies the alarm transmitter 30 that the contact with the living body is released. Then, the alarm transmitter 30 transmits an alarm when it no longer receives the detection signal.

Note that in a case in which the puncture member detachment detection apparatus 2 does not include the alarm transmitter 30, the control unit 222 transmits the detection signal to the alarm transmitter 30 by using a transmission unit (not shown) while the detection signal is being supplied from the rectifying and smoothing circuit 203.

Next, the operation of the heart rate sensor 21 having the above configuration is described. When the heart rate sensor 21 is not in contact with the living body, the permanent magnet 211 does not vibrate, and the output signal of the antenna 215 is approximately 0 V. Accordingly, the output of the rectifying and smoothing circuit 203 is also 0 V, and no detection signal is output.

On the other hand, in a state in which the heart rate sensor 21 is in contact with the living body, the permanent magnet 211 resonates with the vibration of the body surface caused by a heartbeat or respiration, and then the permanent magnet 211 relatively strongly vibrates. Accordingly, the antenna 215 crosses the magnetic flux from the permanent magnet 211, whereby an induced electromotive force is generated in the antenna 215. The frequency of the induced electromotive force is approximately equal to the resonance frequency of the resonance circuit 202, and the resonance circuit 202 amplifies the signal caused by the heartbeat, attenuates the noise frequency, and then outputs it. The rectifying and smoothing circuit 203 generates a detection signal by rectifying and smoothing an AC signal output from the resonance circuit 202. Then, the rectifying and smoothing circuit 203 supplies the detection signal to the control unit 222 in the circuit block 204. The control unit 222 outputs the detection signal to the alarm transmitter 30 while the detection signal is being supplied from the rectifying and smoothing circuit 203.

When the contact of the heart rate sensor 21 with the living body is released, the permanent magnet 211 stops vibrating. Therefore, the control unit 222 stops outputting the detection signal.

Note that FIG. 9 shows a configuration in which the sensor unit 201 and other circuits are arranged side by side, but the arrangement is not limited to this. For example, the sensor unit 201 may be provided on the side of the heart rate sensor 21 that comes into contact with the living body, and other circuits may be provided above the sensor unit 201. By this configuration, it is possible to reduce the area required for the heart rate sensor 21.

As described above, the puncture member detachment detection apparatus 2 according to the second example embodiment of the present invention uses the heart rate sensor 21 as the living body contact detection unit 20. Further, the heart rate sensor 21 is configured to detect that the contact with the living body is released when the heart rate of the living body cannot be measured. Further, the heart rate sensor 21 is configured so that it outputs a detection signal to the alarm transmitter 30 in a state in which the heart rate sensor 21 is in contact with the living body and stops outputting the detection signal when it detects that the contact with the living body is released. Thus, the puncture member detachment detection apparatus 2 according to the second example embodiment of the present invention can cause the alarm transmitter 30 to transmit an alarm when the heart rate sensor 21 detects that the contact with the living body is released.

### Other Example Embodiments

In the second example embodiment, an example in which the heart rate sensor 21 is used as the living body contact detection unit 20 has been described. However, the living body contact detection unit 20 is not limited to this. For example, a temperature sensor 22 or a living body contact communication tag 23 may be used as the living body contact detection unit 20.

When the temperature sensor 22 is used as the living body contact detection unit 20, the temperature sensor 22 may detect that the contact with the living body is released when the temperature becomes equal to or lower than a predetermined temperature. Further, the temperature sensor 22 may detect that the contact with the living body is released by detecting the temperature difference between when the living body is in contact with the living body and when it is not in contact with the living body.

When the living body contact communication tag 23 is used as the living body contact detection unit 20, the living body contact communication tag 23 may detect that the contact with the living body is released when the living body contact communication is interrupted. Note that when the living body contact communication tag 23 is used as the living body contact detection unit 20, the apparatus on the other end of communication with the living body contact communication tag 23 also needs to be in contact with the living body. Therefore, for example, the apparatus on the other end of communication may be provided at a part of a bed that comes into contact with the living body. In this case, the living body contact communication tag 23 can perform living body contact communication with the apparatus on the other end of communication installed in the bed via the living body.

While the present invention has been described with reference to the example embodiments, the present invention is not limited to the aforementioned example embodiments. Various changes that can be understood by those skilled in the art can be made to the configurations and the details of the present invention within the scope of the present invention.

For example, the whole or part of the example embodiments disclosed above can be described as, but not limited to, the following supplementary notes.

### (Supplementary Note 1)

A puncture member detachment detection apparatus comprising:

a puncture member that punctures a living body; and

living body contact detection means for detecting contact with the living body, wherein the living body contact detection means is provided at a part of the puncture member that comes into contact with the living body.

### (Supplementary Note 2)

The puncture member detachment detection apparatus described in Supplementary Note 1, further comprising alarm transmission means, wherein

the alarm transmission means transmits an alarm when the living body contact detection means detects that the contact with the living body is released.

### (Supplementary Note 3)

The puncture member detachment detection apparatus described in Supplementary Note 1 or 2, wherein

the living body contact detection means is a heart rate sensor, and

the heart rate sensor detects that the contact with the living body is released when a heart rate of the living body cannot be measured.

### (Supplementary Note 4)

The puncture member detachment detection apparatus described in Supplementary Note 1 or 2, wherein

the living body contact detection means is a temperature sensor, and

the temperature sensor detects that the contact with the living body is released when a temperature becomes equal to or lower than a predetermined temperature.

(Supplementary Note 5)

The puncture member detachment detection apparatus described in Supplementary Note 1 or 2, wherein

the living body contact detection means is living body contact communication means, and

the living body contact communication means detects that the contact with the living body is released when living body contact communication is interrupted.

### (Supplementary Note 6)

The puncture member detachment detection apparatus described in any one of Supplementary Notes 1 to 5, wherein

the puncture member is a wing-like needle, and

the living body contact detection means is provided at a part of a wing-like part of the wing-like needle that comes into contact with the living body.

### (Supplementary Note 7)

The puncture member detachment detection apparatus described in any one of Supplementary Notes 1 to 5, wherein

the puncture member is an indwelling needle, and

the living body contact detection means is provided at a part of an outer cylinder part of the indwelling needle that comes into contact with the living body.

### (Supplementary Note 8)

A puncture member detachment detection system comprising a puncture member detachment detection apparatus and an alarm transmitter, wherein

the puncture member detachment detection apparatus comprises:
a puncture member that punctures a living body; and
living body contact detection means for detecting contact with the living body,

the living body contact detection means is provided at a part of the puncture member that comes into contact with the living body, and

the alarm transmitter transmits an alarm when the living body contact detection means detects that the contact with the living body is released.

### (Supplementary Note 9)

The puncture member detachment detection system described in Supplementary Note 8, wherein

the living body contact detection means is a heart rate sensor, and

the heart rate sensor detects that the contact with the living body is released when a heart rate of the living body cannot be measured.

### (Supplementary Note 10)

A puncture member detachment detection method comprising:

detecting, by living body contact detection means provided at a part of a puncture member that comes into contact with a living body, contact with the living body; and

notifying an alarm transmitter that the contact with the living body is released when the living body contact detection means detects that the contact with the living body is released.

### (Supplementary Note 11)

A program for causing a processor to execute the following processing of:

detecting, by living body contact detection means provided at a part of a puncture member that comes into contact with a living body, contact with the living body; and

notifying an alarm transmitter that the contact with the living body is released when the living body contact detection means detects that the contact with the living body is released.

This application is based upon and claims the benefit of priority from Japanese Patent Application No. 2018-001118, filed on Jan. 9, 2018, the disclosure of which is incorporated herein in its entirety by reference.

### REFERENCE SIGNS LIST

### 1,2 PUNCTURE MEMBER DETACHMENT DETECTION APPARATUS

### 10 PUNCTURE MEMBER

### 10A WING-LIKE NEEDLE

### 10B INDWELLING NEEDLE

### 11 WING-LIKE PART

### 12 INNER NEEDLE

### 13 OUTER CYLINDER PART

### 20 LIVING BODY CONTACT DETECTION UNIT

### 21 HEART RATE SENSOR

### 22 TEMPERATURE SENSOR

### 23 LIVING BODY CONTACT COMMUNICATION TAG

### 30 ALARM TRANSMITTER

### 100 PUNCTURE MEMBER DETACHMENT DETECTION SYSTEM

What is claimed is:

1. A puncture member detachment detection apparatus comprising:
a puncture member that punctures a living body; and
living body contact detection detector configured to detect contact with the living body, wherein
the living body contact detection detector is provided at a part of the puncture member that comes into contact with the living body.

2. The puncture member detachment detection apparatus according to claim 1, further comprising alarm transmitter, wherein
the alarm transmitter configured to transmit an alarm when the living body contact detection detector configured to detect that the contact with the living body is released.

3. The puncture member detachment detection apparatus according to claim 1, wherein
the living body contact detection detector is a heart rate sensor, and
the heart rate sensor detects that the contact with the living body is released when a heart rate of the living body cannot be measured.

4. The puncture member detachment detection apparatus according to claim 1, wherein
the living body contact detections detector is a temperature sensor, and
the temperature sensor detects that the contact with the living body is released when a temperature becomes equal to or lower than a predetermined temperature.

5. The puncture member detachment detection apparatus according to claim 1, wherein
the living body contact detections detector is living body contact communication unit, and
the living body contact communication unit detects that the contact with the living body is released when living body contact communication is interrupted.

6. The puncture member detachment detection apparatus according to claim 1, wherein
the puncture member is a wing-like needle, and
the living body contact detection detector is provided at a part of a wing-like part of the wing-like needle that comes into contact with the living body.

7. The puncture member detachment detection apparatus according to claim 1, wherein
the puncture member is an indwelling needle, and
the living body contact detection detector is provided at a part of an outer cylinder part of the indwelling needle that comes into contact with the living body.

8. A puncture member detachment detection system comprising a puncture member detachment detection apparatus and an alarm transmitter, wherein
the puncture member detachment detection apparatus comprises:
a puncture member that punctures a living body; and
living body contact detection detector for detecting contact with the living body,
the living body contact detection detector is provided at a part of the puncture member that comes into contact with the living body, and
the alarm transmitter transmits an alarm when the living body contact detection detector detects that the contact with the living body is released.

9. The puncture member detachment detection system according to claim 8, wherein
the living body contact detection detector is a heart rate sensor, and
the heart rate sensor detects that the contact with the living body is released when a heart rate of the living body cannot be measured.

10. A puncture member detachment detection method comprising:
detecting, by living body contact detection detector provided at a part of a puncture member that comes into contact with a living body, contact with the living body; and
notifying an alarm transmitter that the contact with the living body is released when the living body contact detection detector detects that the contact with the living body is released.
